(19) United States
(12) Patent Application Publication
(10) Pub. No.: US 2020/0261495 A1
(43) Pub. Date: Aug. 20, 2020

(54) LIQUID OXYGEN ENCAPSULATION AND METHODS TO ADMINISTER INTRAVASCULAR LIQUID OXYGEN

(71) Applicants: Sivamallikarjuna Reddy AMBATI, Secunderabad (IN); AODH LIFESCIENCES PRIVATE LIMITED, Secunderabad (IN)

(72) Inventor: Sivamallikarjuna Reddy AMBATI, Hyderabad (IN)

(73) Assignees: AODH LIFESCIENCES PRIVATE LIMITED, Secunderabad (IN); Sivamallikarjuna Reddy AMBATI, Secunderabad (IN)

(21) Appl. No.: 16/649,989

(22) PCT Filed: Sep. 23, 2018
(86) PCT No.: PCT/IB2018/057344

§ 371 Date: Mar. 24, 2020

(30) Foreign Application Priority Data
Sep. 24, 2017 (IN) 201741029940

(51) Int. Cl. A61K 33/00; A61K 9/51

(52) U.S. Cl. CPC A61K 33/00; A61K 9/0026; A61K 9/5153

(57) ABSTRACT

The present invention relates to a method for producing encapsulated liquid oxygen to maintain blood homeostasis is disclosed. The composition contains a carrier, encapsulated nano-bubbles with one or more gases, preferably liquid oxygen. The methods and compositions may be administered to a patient to deliver liquid oxygen to the patient's blood supply or directly to a tissue in need of liquid oxygen. The methods and compositions are also meant to eliminate carbon dioxide from blood. The compositions may be administered via injection or as a continuous infusion to a patient in an effective amount to increase oxygen concentration in the patient's blood and/or one or more tissues or organs. The encapsulated liquid oxygen may be administered alone or in combination with other treatments as an adjunctive therapy.

### FIELD OF THE INVENTION

Embodiments of the present invention relate to a polymer encapsulated liquid oxygen composition. More specifically, the embodiments of the present invention relate to a polymer encapsulated liquid oxygen composition comprising oxygen, a polymer, and a cross linking agent.

### BACKGROUND OF THE INVENTION

Approximately there are 34 million critical care admissions recorded annually in India alone, of which 30% of victims die due to the imbalance in blood

Every human cell requires a continuous supply of oxygen to conserve cellular structure and homeostasis. This source of oxygen is mainly provided by hemoglobin, which transports inspired liquid oxygen from the pulmonary capillaries to the tissues and expire carbon dioxide from tissues to pulmonary capillaries. In conditions where a patient's lungs are incapable to transfer adequate amounts of oxygen to circulating erythrocytes, severe hypoxia results and can rapidly lead to severe organ injury and demise. Refurbishment of blood oxygen tension is chief to revival of the majority of pathophysiologic states.

Some clinical states such as lung injury, airway obstruction, asthma, pneumonia and intra-cardiac mixing, exhibit hypoxemia and desaturation refractory to medical efforts to restore levels of oxygen saturation sufficient to limit ischemic injury. Ischemic injury may take place within minutes or seconds due to inadequate oxygen supply. In such conditions the human body may lead to low oxygen tension, can cause end-organ dysfunction, failure and mortality. The capability to elevate oxygenation on the mortality and morbidity from acute hypoxia, in count to a number of other clinical situations.

Even during emergency, patients have to rely on oxygen mask and cannula which are not sufficient to serve the need of liquid oxygen. Moreover, emergency efforts to deliver oxygen to a patient are often inadequate and/or require too long to take effect, either due to lack of an adequate airway or overwhelming lung injury or psychological impact like anxiety, panickiness. This results in irreparable injury to the brain and other vital organs. To initiate rescue therapy in these patients is troublesome and time consuming. There is a need to quickly provide liquid oxygen directly to the blood of patients, thereby inhibiting or lessening irreversible injury due to hypoxemia.

Currently there are traditional attempts to reinstate oxygen levels in patients by using supportive therapy to the patient's respiratory system. Most usually by using the method mechanical ventilation or by Extracorporeal Membrane Liquid oxygenation (ECMO). However, patients with lung injury and intensive care unit patients often face difficulties in exchanging oxygen and carbon dioxide across a damaged respiratory system. This requires clinicians to upsurge ventilator pressures often, this further cause's lung injury and systemic inflammation. Significant morbidity and mortality has been related with ventilator induced lung injury and barotrauma to the lungs which often occurs by inadequate systemic oxygen delivery. A method to non-invasively maintain even small percentages of oxygen delivery can significantly reduce the morbidity of mechanical ventilation and other complexities associated with hypoxia, perinatal asphyxia, breathlessness etc.

US patent application 20050042132 discloses an apparatus for blood oxygenation. The apparatus includes a delivery assembly including an elongated, generally tubular assembly including a central lumen and at least one end placeable within a patient body proximate a tissue site to be treated, the end including an outlet port for the oxygenated blood. However, the apparatus not easy to use and the oxygen used is not encapsulated.

Therefore, in order to overcome the above mentioned drawbacks, there is need to develop a composition and a method to deliver an effective amount of oxygen to a patient to assuage or prevent ischemic injury.

### SUMMARY OF THE INVENTION

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

It is an object of the invention to provide a composition of liquid oxygen nanoencapsulation.

It is another object of the invention to provide a method for preparing liquid oxygen nanoencapsulation.

It is another object of the invention to provide an improved method for delivering liquid oxygen to blood stream of patients, tissues or organs.

It is yet another object of the invention to provide an improved composition for delivering liquid oxygen to patients, tissues or organs.

It is yet another object of the invention to deliver oxygen alone or in combination with other molecules or biologicals to have targeted deliver, controlled release, synergy.

Accordingly, the present invention relates to a polymer encapsulated liquid oxygen composition and a method for delivering and eliminating liquid oxygen and carbon dioxide respectively, directly to/from patient's blood flow and thereby to tissues and organs in respective order for maintaining blood homeostasis.

The underlying object of the present invention is to propose a polymer encapsulated liquid oxygen composition that is easy to use, has no side effects and affordable.

The present invention relates to a polymer encapsulated liquid oxygen composition comprising oxygen, a polymer, and a cross linking agent.

The liquid oxygen is incorporated individually into PEG-PLA nano spheres, and the nanospheres are combined in real-time as and when needed in an aqueous vehicle like saline.

One advantage of the present invention is that to deliver sufficient oxygen through intravenous route for patient in critical condition. Another advantage is targeted delivery of oxygen to solid tumors, which can enhance the efficacy of chemotherapy and radiotherapy.

These and other advantages of the invention will become apparent when viewed in light of the accompanying drawings, examples, and detailed description.

### BRIEF DESCRIPTION OF THE DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 depicts an article of manufacture having features of the present invention;

FIG. 2 depicts an apparatus for making embodiments of the present invention and performing features of the present method.

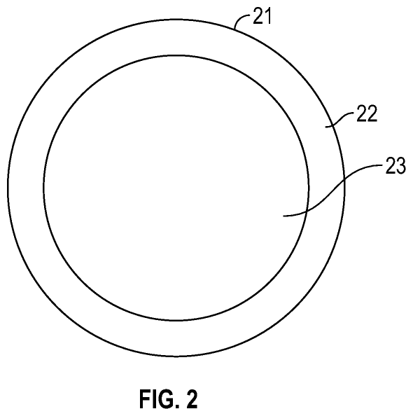

### DETAILED DESCRIPTION

For the purposes of this invention, the following definitions are provided. These definitions are intended to be illustrative and exemplary. They are not intended to restrictively limit the common meaning of the terms to those skilled in the art. These definitions are as follows:

Encapsulation: Encapsulation used herein includes coating, wrapping of one substance with another, enclosing of one substances in another.

Blood gas homeostasis: Blood gas homeostasis means maintaining oxygen and carbon dioxide saturation levels in the blood. Blood gas homeostasis also means maintaining balance of oxygen and carbon dioxide levels to have optimum cellular physiological functioning and metabolism.

Embodiments of the composition and the method will now be described.

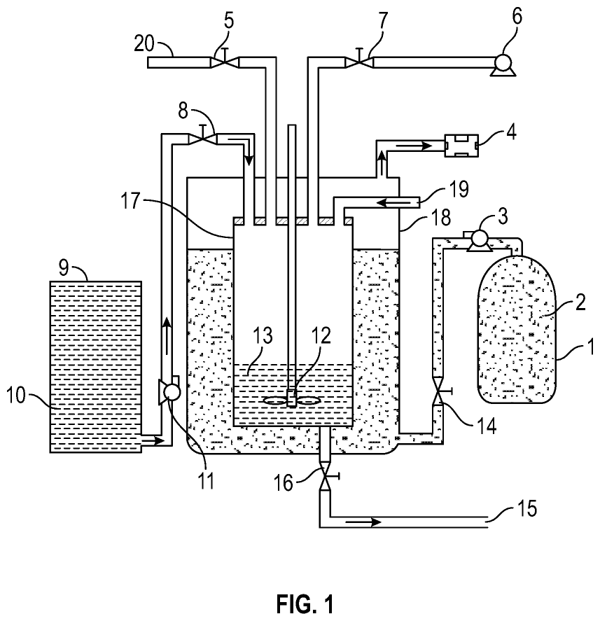

Referring to FIG. 1, an embodiment of the present invention directed to the manufacture of polymer encapsulated liquid oxygen composition is presented. A nano bubble or nano sized polymer encapsulated liquid oxygen, generally designated by the numeral 11 is depicted in cross-sectional view. The sphere 11 has a diameter of about 10 to 5000 nanometers. Although depicted as a sphere, sphere 11 may not be perfect in its geometric form or shape and may have irregularities. Sphere 11 may have particle-like features. The sphere 11 has a shell 15 comprising a biodegradable polymer containing a cannabinoid. The sphere has an interior 17 which comprises the biodegradable polymer, which may or may not be cross linked, and a cannabinoid. A featured cannabinoid is delta-9-tetrahydrocannabinol (delta-9-THC). The shell 15 is cross-linked. To the extent the deareated buffer is incorporated in the sphere 11, the volatile components are substantially lost upon lyophilization. In the present embodiment, the deareated buffer refers to the non-volatilized components of the buffer, for example, one or more sugars which may migrate into the shell 15 and interior 17 upon formation.

The example feature a polymer of poly (D,L-lactide-coglycolide polymer) and polycaprolactone. Referring now to poly (D,L-lactide-coglycolide polymer), this polymer is present in a ratio of 75:25 to 25:75 lactide to glycolide. Other embodiments feature ratios of 60:40 to 40:60 and about 50:50. The poly (D,L-lactide-coglycolide polymer) and polycaprolactone are used in a ratio of about 2 to 1 to 1 to 2 parts by weight lactide-coglycolide to polylactone. These polymers readily form a solution of about one to one parts by weight.

Polymers could be chosen from Dextran (Variants Dextran 400,), PEG, Carboxymaltos, Polyglycolic acid (PGA), Poly(Caprolactone) polymer, Poly(lactic-co-glycolic acid) (PLGA), Polyanhydride, Poly(amide), Poly(Ester amide), Poly phosphoester, Chitosan, L alanine, L-lysine, L-tyrosine, (PLGH (poly (DLLactide-coglycolide)), PNIPAAm [Poly(N-isopropylacrylamide)], pHEMA[Poly 2-hydroxyethyl methacrylate], PAMAM [Poly (amidoamine)], Poly(propyl acrylic acid), Poly(vinyl alcohol), Hyaluronic acid (hyaluronan), Degraded Gelatin, poly-L-aspartic acid, Poly(2-ethyl-2-oxazoline), Icodextrin, Poly malic acid, Poly(meth acrylic acid), Polyorthoester, Polycyanoacrylates, Hydroxypropyl cellulose, chitosan, hyaluronic acid, gelatine, ovalbumin and glycolic polylactic acid, poly vinyl acetate phthalate

A further embodiment of the present invention is directed to a method of making a lyophilized sphere 11 having a diameter of about 10 to 5000 nanometers having a shell 15 comprising a biodegradable polymer containing a cannabinoid. The method comprising the steps of forming a mixture of one or more biodegradable polymers and a cannabinoid in carbon dioxide held under conditions in which carbon dioxide is a supercritical, critical or near critical fluid. The mixture is injected in a stream in a deareated solution comprising a cross-linking agent in a buffer to form one of more spheres having a diameter of 10 to 5000 nanometers. The one or more spheres are lyophilized to form a lyophilized sphere having a diameter of about 10 to 5000 nanometers having a shell comprising a biodegradable polymer containing the cannabinoid.

An apparatus, designated by the numeral 24, for performing an embodiment of the present invention, is depicted in FIG. 1. The apparatus 24 has the following major components: a mixing chamber 22 with a static in-line mixer 12, a solids chamber 26 for containing 33 the supercritical fluid(s), two back pressure regulators 8 and 14, two injector pumps 3 and 11, and sample collection chamber 15 with a valve 16. External to this chamber, two syringe pumps 1 and 27, (Cryo Indian oil), are used for delivery of the supercritical fluid and co-solvent respectively. The outlets of the supercritical fluid and co-solvent syringe 10 pumps 63a and 63b are connected to into the circulation outlet at the entrance of the solids chamber.

There are two take-offs from the high-pressure circulation loop. The first take-off can be achieved by switching the sample valve 37 to allow the circulating stream to flow through a 500 nanoliters-sampling loop. After the sample is trapped, the sampling loop is flushed with a liquid solvent such as acetone to collect the polymer dissolved in 500 nanoliters of supercritical, critical or near critical carbon dioxide with or without co-solvent such as an alcohol. The second take-off from the high-pressure circulation loop is at the top of the mixing chamber 31. This take-off is connected to the inlet of static in-line mixer 39. The feed syringe pump for a cannabinoid rich stream is connected to the inlet of the static in-line mixer 39.

The apparatus 21 is maintained as a closed system. The entire apparatus up to the backpressure regulators 41a and 41b is designed to operate up to 5,000 psig and 60° C. The apparatus 21 is cleaned in-place by washing with a series of solvents including bleach, caustic and dilute hydrochloric acid, and then sterilized in-place with an ethanol/water (70/30) mixture.

Methods

Method 1

Oxygen nanospheres are formed with temperatures maintained under Liquid nitrogen or Near-Critical Propane. Oxygen nanospheres were formed with 50:50 PLGA obtained from Sigma Chemicals in the cryogenic encapsulation apparatus, FIG. 1, running in the continuous mode. The polymer nanospheres/nanospheres were formed by injecting the polymer solution into distilled water. The polymer solution is stirred at up to 50000 rpm in the encapsulation chamber at temperature ranging −100° degrees to 25° C. and pressure 1 atm to 25 atm, by adding liquid oxygen through liquid oxygen inlet. The liquid oxygen is maintained at a concentration of 5 to 65% in the encapsulation chamber. 2% Ethanol is used as cosolvent. 0.01 to 1% of aluminium chloride is added as crosslinking agent. The stirring is continued for up to 8-12 hours. The resulting mixture is freeze dried to obtain oxygen nanospheres. The resulting product was observed under a light nanoscope, and the particle sizes were measured in a dynamic light scattering.

### Example 1

Liquid Oxygen in Polymer Nanospheres:

Experiments were performed to encapsulate liquid oxygen in 50:50 PLGA polymer nanospheres formed by supercritical carbon dioxide and propane. In this liquid oxygen circumvened with supercritical carbon dioxide with 10% (v/v) cosolvent ethanol, and near-critical propane in the presence of PLGA. The pressure and temperature were around 3,000 psig and −30 to 35° C. respectively. The method in example 1 is repeated.

The cosolvent is selected from the list of Methylene chloride, ethylene chloride, carbon tetrachloride, chloroform, diethyl ether, methyl ethyl ether, hexane, heptane, cyclohexane, petroleum ether, benzene, nitro methane, carbon disulfide, toluene, methyl acetate, ethyl acetate, butyl acetate, amyl acetate, methyl formate, ethyl formate, butyl formate, Toluene-ethanol, methyl ethyl ketone, Toluene-ethanol-ethyl acetate, acetone, benzene, Toluene, Nitropropane, dioxane, Tetrahydro-naphthalene (K) Petroleum distillate, Polybutadiene, Ethanol, Phenol-methyl siloxane, Methacrylic polymer, Ethylene glycol, glycerine, ethanol, methanol, dichloromethane, methylene chloride

Method 2:

Polymer encapsulated liquid oxygen nanospheres are formed by injecting the polymer-rich, cannabinoid laden carbon dioxide fluid with one or more entrainers such as an alcohol into a 1% polyvinyl alcohol (PVA) deareated buffer solution. The buffer preferably contains a sugar such as sucrose. Other media such as high concentration sucrose solutions to aid in particle stability during lyophilization, liquid nitrogen for freezing the particles and phosphate-buffered saline at physiological pH as a control can be used. Other collection media parameters that impact the size and uniformity of the nanospheres are temperature and pressure. Lower temperatures are much more favorable for polymer and liquid oxygen stabilities. Operating pressure as well as pressure in the particle formation chamber control the size and uniformity of bubbles formed and nanospheres generated. The pressure in the encapsualation chamber can be varied from the vapor pressure of the near supercritical, critical or near critical fluid at the temperature of the medium to atmospheric pressure.

Method 3:

Preparation of PLGA Nano Sphere Containing 40% by Weight of Liquid Oxygen

An O/W emulsion was prepared using liquid oxygen as the oily phase in which 5% of poly-lactic-co-glycolic acid (PLGA) was dissolved together with liquid oxygen. The water phase is a phosphate buffer at pH 7 with 0.8% of poly-vinyl alcohol (PVA) (all the percentages are expressed by weight).

The emulsion was prepared with a 20:80 ratio by using a high speed homogenizer operating at 2900 rpm for 3 min. The emulsion was mixed with compressed CO₂ to obtain the 0.5-10% by weight of the dispersant phase in a static mixer at 80 bar 38° C. and in these conditions it remained stable. Then, the emulsion was fed to the packed column. CO₂ was taken in liquid form from a cylinder and sent to a pump that generates pressures in this case between 70 and 120 bar, preferably 80 bar. Simultaneously, the column temperature was set between −35° C. and 38° C. In this particular case, the temperature control is important because the polymer used has a glass transition temperature of 40° C.

After about 30 minutes the pressure and the temperature reached the steady state conditions. At this point, the expanded emulsion was pumped from the top of the column. The emulsion flow rate varied from 1/10 to 3/10 of the CO₂ flow rate. As the emulsion flowed along the column, compressed CO₂ extracted the ethyl acetate, inducing the formation of polymeric nanospheres containing the active ingredient. The so-formed expanded suspension gathered at the bottom of the column, with a content of ethyl acetate residue of less than 30 ppm. The collected suspension was washed with distilled water to remove the surfactant by ultracentrifugation at 8000 rpm for 10 min at 4° C. Finally, the material was dried.

Method 4

Preparation of Nanospheres Wherein the Starting Polymer is a Crosslinked Polysaccharide of Hyaluronic Acid (ACP)

A hyaluronic acid derivative, wherein 10% of the carboxy groups of hyaluronic acid are bound with inter- or intramolecular hydroxy groups and the remaining part is salified with sodium, is dissolved in an aprotic solvent such as dimethylsulfoxide (DMSO), at a concentration varying between 0.1 and 5% in weight, generally 1% w/w. The procedure described in method 1 is then performed. The mean particle size is 0.4μ.

Method 5

Preparation of Nanospheres Wherein the Starting Polymer is an Ester of Alginic Acid (ALAFF)

A derivative of alginic acid, wherein all the carboxy groups of alginic acid are esterified with benzyl alcohol, is dissolved in an aprotic solvent, such as dimethylsulfoxide (DMSO), at a concentration varying between 0.1 and 5% in weight, generally 1% w/w. The procedure described in method 1 is then performed. The mean particle size is 0.7μ

Method 6

Preparation of Nanospheres Wherein the Starting Polymer is an Ester of Pectinic Acid

A derivative of pectinic acid, wherein all the carboxy groups are esterified with benzyl alcohol, is dissolved in an aprotic solvent, such as dimethylsulfoxide (DMSO), at a concentration varying between 0.1 and 5% in weight, generally 1% w/w. The procedure described in Example 1 is then performed. The mean particle size is 0.9μ.

Optimum oxygen nanospheres formation, size and liquid oxygen encapsulation depend on the ratio of polymer to liquid oxygen in the sample collection chamber(s). This ratio depends on the flowrate of the liquid oxygen-rich stream and its concentration, and the flowrate of the polymer-rich supercritical, critical or near critical fluid stream and its concentration (which is defined by polymer solubility at operating conditions). The polymer:liquid oxygen ratio can be varied from 100:1 to 1:1. Should there be problematic aggregation of the polymer nanospheres after their formation, the agglomeration is broken by utilizing liquid oxygen nanospheres.

Stability Studies:

Shelf stability studies were conducted with Δ9-THC, Δ9-THC polymer nanospheres and formulations of the aforementioned. The following tests were performed: (i) physical appearance; and (ii) Δ9-THC content and integrity. Statistical analysis of the data sets were performed using SYSTAT®.

Encapsulation Efficiency:

The loading efficiency of Δ9-THC in polymer nanospheres were determined by dissolving a known amount of nanospheres in a 90% acetonitrile aqueous solution. The amount of Δ9-THC were determined by HPLC assay, and the loading efficiency was calculated based on weight percent.

Applications:

The technology can be used to deliver oxygen alone or in combination with other molecules or biologicals to have targeted deliver, controlled release, synergy etc.

Although the invention has been described with reference to exemplary embodiments, it is not limited thereto. Those skilled in the art will appreciate that numerous changes and modifications may be made to the embodiments of the invention and that changes and modifications may be made without departing from the true spirit of the invention.

### LIST OF REFERENCE NUMERALS

1—Cryogenic cylinder,
2—Liquid nitrogen,
3—Pump,
4—Pressure outlet,
5—valve,
6—vacuum pump,
7—valve,
8—liquid oxygen valve,
9—cryogenic cylinder,
10—liquid oxygen,
11—liquid oxygen pump,
12—homogeniser,
13—homogenized mixture,
14—liquid nitrogen pump,
15—sample collection outlet,
16—sample collection valve,
17—process chamber,
18—Temperature maintenance chamber,
19—polymer inlet,
20—raw material inlet.

1. A polymer encapsulated liquid oxygen composition comprising oxygen, a polymer and a cross linking agent.

2. The polymer encapsulated liquid oxygen composition as claimed in claim 1, wherein the polymer is selected from Dextran (Variants Dextran 400), PEG, Carboxymaltos, Polyglycolic acid (PGA), Poly(Caprolactone) polymer, Poly(lactic-co-glycolic acid) (PLGA), Polyanhydride, Poly(amide), Poly(Ester amide), Poly phosphoester, Chitosan, L alanine, L-lysine, L-tyrosine, (PLGH (poly (DLLactide-coglycolide)), PNIPAAm [Poly(N-isopropylacrylamide)], pHEMA[Poly 2-hydroxyethyl methacrylate], PAMAM [Poly (amidoamine)], Poly(propyl acrylic acid), Poly(vinyl alcohol), Hyaluronic acid (hyaluronan), Degraded Gelatin, poly-L-aspartic acid, Poly(2-ethyl-2-oxazoline), Icodextrin, Poly malic acid, Poly(meth acrylic acid), Polyorthoester, Polycyanoacrylates, Hydroxypropyl cellulose, chitosan, hyaluronic acid, gelatine, ovalbumin and glycolic polylactic acid, poly vinyl acetate phthalate.

3. The polymer encapsulated liquid oxygen composition as claimed in claim 1, wherein liquid oxygen is wrapped in the polymer sphere.

4. The polymer encapsulated liquid oxygen composition as claimed in claim 1, wherein the composition is in the form of powder.

5. The polymer encapsulated liquid oxygen composition as claimed in claim 1, wherein the composition is used for intravenous oxygen delivery.

6. A method for preparing polymer encapsulated liquid oxygen composition, the method comprising the steps of:
a) formation of liquid oxygen nanospheres with 50:50 PLGA obtained in the cryogenic encapsulation apparatus;
b) injecting the polymer into distilled water to form the polymer solution;
c) the polymer solution is stirred at up to 50000 rpm in the encapsulation chamber at temperature ranging −100° C. to 25° C. and pressure 1 atmosphere to 25 atmosphere, by adding liquid oxygen through liquid oxygen inlet apparatus and 2% ethanol is used as cosolvent;
d) the liquid oxygen is maintained at a concentration of 5% to 65% in the encapsulation chamber;
e) 0.01 to 1% of aluminium chloride is added as crosslinking agent;
f) the stirring is continued for up to 8-12 hours;
g) the resulting homogenized mixture is freeze dried to obtain oxygen nanospheres in the form of powder

7. The polymer encapsulated liquid oxygen composition as claimed in claims 1 to 6 is used for treatment of hypoxia, blood gas homeostasis, sickle cell anemia, carbon monoxide poisoning, traumatic brain injury or of stroke, cancer, alzheimers disease.
